(19) United States
(12) Patent Application Publication
(10) Pub. No.: US 2024/0037740 A1
(43) Pub. Date: Feb. 1, 2024

(54) METHOD AND SYSTEM FOR AUTOMATIC IHC MARKER-HER2 SCORE

(71) Applicant: Applied Materials, Inc., Santa Clara, CA (US)

(72) Inventors: Sumit Kumar JHA, Bangalore (IN); Gursewak SINGH, Bangalore (IN); Rithika CARIAPPA, Bangalore (IN); Kiran Rangaswamy AATRE, Bangalore (IN)

(21) Appl. No.: 18/360,074

(22) Filed: Jul. 27, 2023

(30) Foreign Application Priority Data
Jul. 28, 2022 (IN) 202241043243

(51) Int. Cl. G06T 7/00; G06V 10/82; G06V 10/26; G16H 50/30

(52) U.S. Cl. CPC G06T 7/0012; G06V 10/82; G06V 10/267; G16H 50/30; G06T 2207/30024; G06T 2207/10056; G06T 2207/30068; G06T 2207/10024

(57) ABSTRACT

Methods and systems for generating a predictive HER2 score using machine learning models are disclosed. An example method generally includes identifying a plurality of nuclei and membrane segments in regions of interest in an input image using a first machine learning model. For the plurality of nuclei and membrane segments identified in the input image, a plurality of features are extracted and classified into one of a plurality of feature categories. Using a second machine learning model, a predictive HER2 score indicating the likelihood of whether a stained tissue sample captured in the input image is HER2 positive or HER2 negative is generated based on the classification assigned to the plurality of extracted features associated with the plurality of segments.

### RELATED APPLICATIONS

This application claims benefit of and priority to Indian Patent Application No. 202241043243, filed Jul. 28, 2022, the entire contents of which are incorporated herein by reference.

### BACKGROUND

### Field

Embodiments of the present disclosure generally relate to cell tissue sample scoring using image data, and more particularly to a method and system for the automatic generating of IHC HER2 score predictions for images of IHC stained tissue samples.

### Description of the Related Art

Human epidermal growth factor receptor 2 (HER2) is a protein found on cells that has been found to be a key component in regulating cell growth. However, when the HER2 protein is altered in mutated cells such as cancer cells, extra HER2 protein receptors may be produced. The over-expression of HER2 protein receptors causes increased cell growth and reproduction. HER2 protein overexpression in cancer cells has been found to be a predictive marker for treatments based on HER2 targeted therapy. Based on this finding, multi-stain based HER2 testing have been developed for invasive cancers to assist physicians in decisions concerning treatment. HER2 testing has therefore become a routine practice for screening of diagnosed cancer cells in pathology.

In the screening of breast cancer cells, the HER2 testing results are represented by a HER2 “score” ranging from IHC 0 to IHC 3. HER2 scores for analyzed tissue samples are then utilized to assess a HER2 status diagnosis (HER2 positive or HER2 negative) for the patient. Traditionally, the process of assessing a HER2 “score” for a cell tissue sample is purely based on the visual analysis of IHC stained tissue samples by an examining pathologist or physician. The process is time consuming and results are often inconsistent.

Therefore there is a need in the art for improved systems and methods for automatic IHC HER2 scoring of cell tissue samples to assist with treatment of breast cancer, such as providing HER2 scores predictions of analyzed cancer tissue samples to assist with HER2 status diagnosis.

### SUMMARY

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

In an embodiment, the present disclosure provides a computer-implemented system and method for generating a predictive HER2 score for an image of an IHC stained cancer tissue sample. According to certain embodiments of the present disclosure, the HER2 score prediction system presented herein is designed to provide a prediction of a regional HER2 score for each contiguous region of IHC stained cancer tissue cells in an image, as well as to provide an overall HER2 score prediction for the sample as a whole captured in the image. The overall HER2 score prediction is based on the one or more regional HER2 score predictions generated.

In an embodiment, the method generally includes designating one or more regions of interest in a patient image comprising an IHC stained tissue sample and identifying a plurality of segments in the patient image using a first machine learning model, wherein each of the plurality of segments comprise nuclei or membrane in the one or more regions of interest. A plurality of features is extracted from the plurality of segments identified in the input image by the first machine learning model. The plurality of features may then be classified into one of a plurality of feature categories. Using a second machine learning model, a predictive HER2 score is generated based on the classification assigned to the plurality of extracted features associated with the plurality of segments. The predictive HER2 score may indicate the likelihood of whether a stained tissue sample captured in the input image is HER2 positive or HER2 negative.

Still further embodiments provide a computer-implemented method for training a predictive HER2 tissue scoring model to generate a predictive HER2 score for an image of a stained tissue sample using machine learning models. An example method generally includes receiving a training data set including a plurality of images of stained tissue samples. In one aspect, the plurality of images of stained tissue samples in the training data may be labeled to identify segments of membrane and nuclei captured in the plurality of images. In another aspect, features of the membrane and nuclei labeled in the plurality of images may be classified into one of a plurality of feature categories. A first machine learning model is trained to classify segments of an input image as nuclei or membrane based on the training data set. A second machine learning model is trained to generate a predictive HER2 score for the input image based on the training data set. The predictive HER2 score indicating the likelihood of whether the stained tissue sample captured in the input image is HER2 positive or HER2 negative.

Other embodiments provide processing systems configured to perform the aforementioned methods as well as those described herein; non-transitory, computer-readable media comprising instructions that, when executed by one or more processors of a processing system, cause the processing system to perform the aforementioned methods as well as those described herein; a computer program product embodied on a computer readable storage medium comprising code for performing the aforementioned methods as well as those further described herein; and a processing system comprising means for performing the aforementioned methods as well as those further described herein.

The following description and the related drawings set forth in detail certain illustrative features of one or more embodiments.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments of the present disclosure and are therefore not to be considered limiting of its scope, and may admit to other equally effective embodiments.

FIG. 1 depicts a schematic diagram of an example system for generating a predictive HER2 score for a stained tissue sample image, according to certain embodiments.

FIG. 2 depicts a schematic diagram of an example process for generating a predictive HER2 score, according to certain embodiments.

FIG. 3 depicts a schematic diagram of an example deep-learning image analysis system, according to certain embodiments.

FIG. 4 depicts a schematic diagram showing an example of the training and use of a deep-learning machine learning model, according to certain embodiments.

FIG. 5 depicts a schematic diagram showing an example of the training and use of a machine learning model, according to certain embodiments.

FIG. 6A depicts a flow diagram of an example method for training and use of the system in FIG. 1 with the processes of FIGS. 2, 4, and 5, according to certain embodiments.

FIGS. 6B and 6C depict flow diagrams of example methods for use of the system in FIG. 1 to generate a predictive HER2 score, according to certain embodiments.

FIG. 7 depicts a schematic diagram of an example processing system for generating a predictive HER2 score, according to certain embodiments.

FIGS. 8A-8C depict an example User Interface Visualization Output, according to certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Traditionally, one of the primary laboratory methods used to determine HER2 status of breast cancer cells is an immunohistochemistry (IHC) assay staining analysis to view the level of HER2 protein expression in a tissue sample. IHC is generally used in histology to detect the presence of specific protein markers that may correspond with certain tumors. For uniformity in accuracy and reproducibility of HER2 testing in breast cancer cells, the American Society of Clinical Oncology (ASCO)/College of American Pathologists (CAP) jointly released instructions and HER2 scoring guidelines for HER2 testing of diagnosed breast cancer patients. The ASCO/CAP guidelines for IHC HER2 scoring include recommendations that HER2 status be initially assessed by IHC using a semi-quantitative scoring system. As such, the prevalent practice in the industry for HER2 testing has been to use IHC as a screening test and FISH as a confirmation test for HER2 IHC equivocal cases.

To assess an IHC HER2 score for tissue samples using IHC analysis, stained tissue samples from IHC assays are placed on a microscope slide to form a static sample for viewing through a microscope or other microscopic viewing device. Images of the static sample may be obtained to generate a whole slide image (WSI) file of the static sample for storing and later review of the static sample. The WSI of the stained tissue sample may subsequently be reviewed and evaluated by a pathologist, who then makes objective and subjective decisions using ASCO/CAP guidelines to generate an IHC HER2 score for the analyzed and observed stained tissue sample in the WSI. Pursuant to ASCO/CAP guidelines, IHC HER2 scores for invasive breast cancer specimens range from IHC0 to IHC3+. For the determination of HER2 status using IHC HER2 scoring results, evaluated stained specimens with scores of IHC3+ are regarded as HER2 positive, specimens with IHC0 or IHC1+ scores are regarded as HER2 negative, and cases with IHC2+ are considered equivocal for HER2 protein expression requiring further testing and confirmation using a second IHC or FISH test.

IHC assays detect HER2 protein overexpression using monoclonal or polyclonal antibodies that bind to the HER2 protein. The HER2 scoring method disclosed herein assumes that the IHC assays used are standardized with rigorous quality control. Examples of US Food and Drug Administration (FDA) approved methods for HER2 assessment using IHC assay kits include HerceptTest™ (DAKO, Glostrup Denmark) and HER2/neu (4B5) rabbit monoclonal primary antibody (Ventana, Tucson, Arizona). Such kits are known to contain well-standardized, high-quality reagents of known specificity and sensitivity. The standardization applies to all parameters of testing including all aspects of pre-analytical tissue-sample handling, the type and duration of fixation, tissue processing, assay performance, interpretation, and reporting.

However, there is no universal gold standard that can accurately measure the expression level of HER2 protein. The IHC assay cannot quantitatively measure the expression level of HER2 protein. Instead, the scoring method for HER2 expression based on ASCO/CAP guidelines is dependent on the semi-quantitative visual evaluation of the cancer cell staining pattern by the examining pathologist who determines a HER2 score based on the observed membrane staining in the stained tissue sample. Specifically, the static tissue sample is evaluated based on the (1) completeness of the membrane staining observed on the stained cancer cells, if any, the (2) intensity of the membrane staining, if present, and (3) the % of cancer cells the staining, if any, is observed in. Thus, the subjectivity of the pathologist may be involved resulting in the false-positive or false-negative determination leading to a low reproducibility of the determination.

To overcome these and other shortcomings, according to certain embodiments of the HER2 scoring system and methods disclosed herein, the HER2 score prediction system is designed to provide a HER2 score prediction for images of IHC stained breast cancer tissue samples to assist physicians with assessing a HER2 status diagnosis.

In certain embodiments, the HER2 score prediction system described herein may use various algorithms or artificial intelligence (AI) models, such as deep learning or machine-learning models, trained based on images of IHC stained and confirmed breast cancer cells to provide automatic real-time HER2 score predictions for images of IHC stained breast cancer tissue samples. Due to ASCO/CAP guidelines for IHC-HER2 scoring, certain aspects are directed to algorithms and/or machine-learning models designed to detect nuclei and membrane in images of IHC stained tissue samples to analyze and use the IHC staining of each nuclei and membrane in a cancer tissue sample to generate a HER2 score prediction for the respective analyzed sample. The algorithms and/or machine-learning models may be used in combination with one or more other image processing software or tools configured to extract and classify features from the analyzed image relevant to ASCO/CAP HER2 scoring guidelines. In particular, the algorithms and/or machine-learning models may take into account parameters, such as the intensity and completeness of the staining of the nuclei and membrane in the stained tissue sample being analyzed. Based on these parameters, the algorithms and/or machine-learning models may provide a prediction of a HER2 score for the stained tissue sample in the analyzed image.

According to certain embodiments, prior to deployment, the machine learning models are trained with training data, e.g., labeled nuclei and membrane image segments. As described in more detail herein, a human viewer may prepare training data by reviewing and labeling each image segment of a WSI depicting an IHC stained breast cancer tissue sample to indicate and identify segments of stained nuclei and membrane depicted in the WSI. Once every image segment of the WSI is reviewed and labeled as depicting nuclei, membrane, or neither, the labeled image segments may be used to train a deep-learning machine model, such as a convolutional neural network (CNN), to detect nuclei and membrane in whole slide images.

In some aspects, in order to train the machine learning model to be scanner-agnostic, the image segments used as training data may be augmented with varying contrast, brightness, blurriness, sharpness, cropping, cutting and mixing, elastic transformation and colors to accommodate for variations in the WSI due to differences in scanners, scopes, and cameras used to generate the WSI of the stained tissue sample. The image segments for training may also be rotated and inputted in varying orientations to accommodate for varying positions and orientations corresponding to placement of the tissue sample slide when the WSI is captured and generated.

Once nuclei and membranes in the WSI are detected by the deep-learning machine learning model, features related to the detected nuclei and membrane are then extracted and classified based on HER2 scoring relevant factors in ASCO/CAP guidelines for use in generating a HER2 score prediction. In certain embodiments, the data associated with the extracted and classified features that correspond to the analyzed nuclei and membrane segments may be further used to train another machine learning model, such as a Random Forest or Support Vector Machine model, to classify a HER2 score for the analyzed stained tissue sample depicted in the WSI. As described in more detail herein, the training features data comprising the extracted and classified features data corresponding to labeled nuclei and membrane image segments and may be provided in a form of a dataset including data records. Data labeling is the process of adding one or more meaningful and informative labels to provide context to the data for learning by the machine learning models. Each data record is featurized (e.g., refined into a set of one or more features, or predictor variables) and labeled based on the extracted and classified nuclei and/or membrane features relevant to HER2 scoring. In certain embodiments, each data record is labeled with one or more nuclei and/or membrane feature, a classification of the respective feature based on the nuclei and/or membrane depicted in the image segment. The features associated with each data record may be used as input into the second machine learning model, and the generated output may be compared to label(s) assigned to each of the data records (e.g. corresponding HER2 score output for the respective training nuclei/membrane image). The models may compute a loss based on the difference between the generated output and the provided label(s). This loss can then be used to modify the internal parameters or weights of the models. By iteratively processing features associated with each data record corresponding to each historical patient, the models may be iteratively refined to generate accurate predictions of HER2 scores for images of stained tissue samples based on the features associated with the staining of the nuclei and membrane in the image.

Once the machine learning models are trained, the models may be used as part of the HER2 score prediction system described herein to provide regional and overall HER2 score predictions for IHC stained tissue samples captured in a new WSI. In certain aspects, the HER2 scoring system may comprise supporting image visualizations the generated HER2 score prediction is based on to assist physicians and pathologists in their own determination of a final HER2 score and HER2 status diagnosis.

In one aspect, in addition to generating a HER2 score prediction for the stained tissue sample in the WSI, the second machine learning model may also generate one or more regional HER2 scores for WSI input images comprising one or more regions of homogenous and contiguous invasive cell populations (Regions of Interest) depicted in the WSI. In certain embodiments, each Region of Interest (ROI) in the WSI is classified with a regional HER2 score prediction along with an overall HER2 score prediction outputted based on the various regional HER2 score predictions generated for the WSI.

The use of machine learning models and/or algorithms for predicting IHC HER2 scores for images of stained tissue samples enables consistent, real-time assistance through provision of HER2 score predictions to assist physicians and pathologists during their own HER2 score assessment and diagnosis of the stained tissue sample in the WSI. In addition, human subjectivity can be minimized during the scoring process, thereby producing better patient outcomes.

Although example embodiments are presented with respect to a system and method for automatic IHC HER2 scoring of breast cancer cells, the methods and systems presented herein may generally be applied to the scoring of other IHC stained tissue cells.

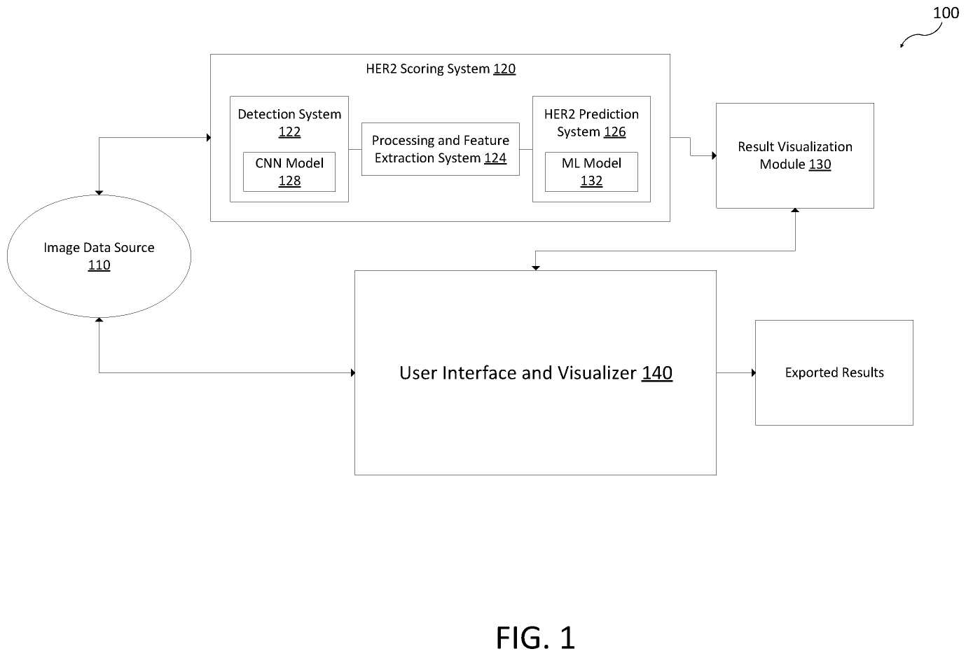

FIG. 1 is a schematic diagram showing an example automatic HER2 scoring system 100 for generating a predictive HER2 score for a stained tissue sample image. In an embodiment, system 100 includes an image data source 110, a HER2 scoring system 120, a result visualization module 130, and a user interface and visualizer 140. Image data source 110 may include a microscope, a light microscope, an electron microscope, and the like for viewing the microscopic structures of the stained tissue sample. The image data source 110 may also include a camera for capturing images of the stained tissue sample as WSI input data 102 for use by the system 100.

In the embodiment shown, the HER2 scoring system 120 in system 100 processes the WSI input data 102 received from the image data source 110 and outputs the predictive HER2 score for the inputted WSI input data 102. The HER2 scoring system 120 includes a Detection System 122, a Processing and Feature Extraction Module 124, and a HER2 prediction system 126. Detection System 122 may be implemented with a deep-learning framework having a convolutional neural network (CNN) 128. Deep-learning models using a CNN can be used for image analysis. As such, the deep-learning framework in Detection System 122 may be trained and utilized to analyze the WSI input data 102 of stained tissue samples to detect each of the nuclei and membrane depicted in the WSI.

The Detection System 122 processes the WSI input data 102 and outputs the processed data as a WSI detected data 104 to the Processing and Feature Extraction Module 124. The WSI detected data 104 includes portions of the images from the image data source 110 being classified by the Detection System 122 as depicting nuclei and/or membrane. The Processing and Feature Extraction Module 124 then prepares and analyzes the WSI detected data 104 for scoring, including reassembling portions of the images in the WSI detected data 104 together and extracting and classifying one or more of a plurality of features from the nuclei and membrane detected by the Detection System 122 in the WSI detected data 104. The Processing and Feature Extraction Module 124 then outputs a WSI extracted data 106 to the HER2 prediction system 126 for classification of the WSI extracted data 106 with one or more regional HER2 score predictions 108. In one aspect, a regional HER2 score prediction 108 may be generated for each group of homogenous and contiguous invasive cell population captured in the WSI.

In the example shown, the HER2 prediction system 126 includes a machine learning (ML) model 132. According to certain embodiments, detection system 122 may alternatively implement one or more other types of machine learning models, including without limitation, a graph neural network, recurrent neural network, capsule neural network, or other deep-learning model capable of learning to recognize and detect images, or portions of images. According to certain embodiments, ML Model 132 may include one or more of a random forest walk, a support vector machine, a decision tree, a convolutional neural network, or other ML model capable of classifying images, or portions of images.

Once the one or more regional HER2 score predictions 108 are obtained for the WSI input data 102, the HER2 scoring system 120 outputs the one or more regional HER2 score predictions 108 to the result visualization module 130 in which additional processing of the WSI input data 102 is performed with one or more of the one or more regional HER2 score predictions 108, the WSI detected data 104, or the WSI extracted data 106 prior to generating a visual output to the User Interface and Visualizer 140. Once the visual output is generated for the User Interface and Visualizer 140, pathologists and physicians may use the User Interface and Visualizer 140 to review the one or more regional HER2 score predictions 108, an overall HER2 score prediction 109 for the WSI based on the one or more regional HER2 score predictions 108 if there are more than one regional HER2 score predictions. In one aspect, the User Interface and Visualizer 140 may generate and provide a region mask 114 overlaid on the WSI showing each of the designated ROIs analyzed by the HER2 scoring for generating the HER2 score predictions. In another aspect, the User Interface and Visualizer 140 may generate a heat mask 116 showing each of membrane and nuclei detected in each of the ROI in the WSI for reference with the one or more regional HER2 score predictions 108 outputted by the HER2 Prediction System 126. The results and summary from the User Interface and Visualizer 140 may be further exported to a portable format or shared to third-party healthcare management systems for further review.

Although HER2 scoring system 120 is depicted with the Detection System 122, the Processing and Feature Extraction Module 124, and the HER2 prediction system 126, according to certain embodiments, one or more of these may be physically located remotely from system 100 and accessed via a network.

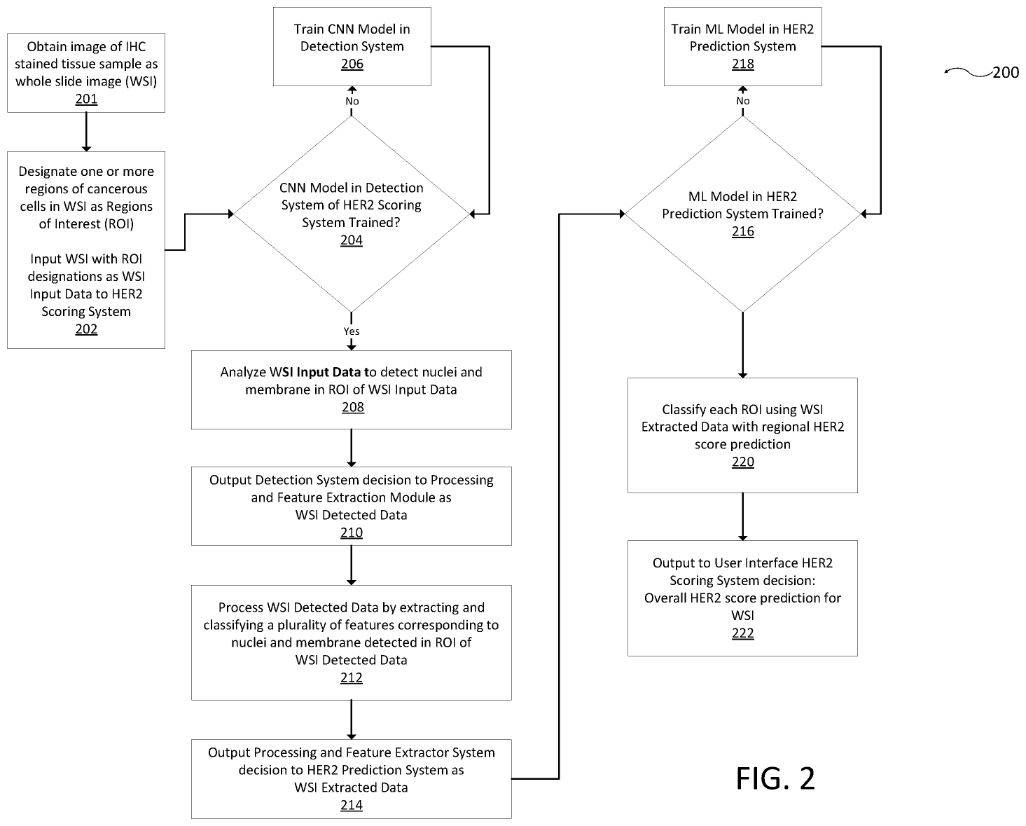

FIG. 2 depicts a diagram of an example process flow 200 for operations of IHC HER2 Scoring using system 100, according to certain embodiments. At block 201, a WSI—whole slide image of IHC stained breast cancer tissue sample is obtained for analysis by the system 100.

In one aspect, the WSI image may comprise both cancerous and normal cell populations. In step 202, each of the one or more regions of cancerous cell populations depicted in the WSI are designated as Regions of Interest (ROI) in the WSI for analysis by the HER2 scoring system 120. The WSI may comprise one or more ROI's depending on the number of regions of contiguous cancerous cell populations. In an embodiment, the one or more ROIs in the WSI may be designated manually by a user prior to deployment of the HER2 scoring system 120. Alternatively, software, algorithms, or artificial intelligence (AI) models, such as deep learning or machine-learning models, may be used to analyze and designate the one or more ROIs in the WSI. The WSI with ROI designations is then inputted as WSI input data 102 into the Detection System 122 of HER2 scoring system 120 for detection of nuclei and membrane in each of the ROI in the WSI.

At block 204, the Detection System 122 determines if a first trained machine learning is available for detecting nuclei and membrane in the WSI input data 102. In the embodiment shown, the first machine learning model may be CNN model 128 from system 100. If a trained version of CNN model 128 is not available, the process proceeds to block 206 where CNN model 128 is trained with labeled training data. Otherwise, the process proceeds to block 208.

At block 208, the WSI input data 102 is analyzed with the trained CNN model 128, providing at block 210 a WSI Detected Data 134 output identifying segments of nuclei and membrane captured in images of the WSI. In an example, since the image of the WSI input data 102 may be too large to be analyzed by the CNN model 128 all at once, the CNN model 128 may instead analyze the image of the WSI input data 102 in segments. Specifically, to preserve the spatial relationships between the pixels in the images of the WSI input data 102, the CNN model 128 may create matrices called filters (or kernels) which the CNN model 128 will slide over the matrix of pixels in the images of the WSI input data 102. In certain embodiments, in analyzing the WSI input data 102, the CNN model 128 may therefore analyze the WSI input data 102 in 512×512-pixel image segments and classify each image segment as depicting either nuclei and/or membrane. Image segments in which neither nuclei nor membrane are detected may be disregarded by the CNN model 128 as background. The WSI Detected Data 134 outputted by the CNN model 128 may therefore include various classified 512×512-pixel image segments of nuclei and membrane captured in the WSI input data 102. In an embodiment, the CNN model 128 may alternatively analyze and output the WSI Detected Data 134 in 256×256-pixel image segments, 96×96-pixel image segments, or any other sized image segments capable of being processed by a neural network.

At block 212, the WSI Detected Data 134 is then provided to the Processing and Feature Extraction Module 124 for further processing and analysis in preparation for the HER2 Prediction System 126. In block 214, the WSI Detected Data 134 is processed by the Processing and Feature Extraction Module 124 and outputted to the HER2 Prediction System 126 as a WSI Extracted Data 106.

In one aspect, the Processing and Feature Extraction Module 124 may stitch or reassemble the plurality of classified 512×512-pixel nuclei and membrane image segments outputted from the CNN model 128 back together (“Reassembled WSI”) such that stitched image segments resemble the original stained tissue sample image of the WSI input data 102. The reassembling of the classified nuclei and membrane image segments from the CNN model 128 allows for each of the detected nuclei and membrane in the WSI to be viewed as a whole in relation to each of the ROI in the original tissue sample image captured. After the plurality of classified image segments are stitched together, the Extraction Module 124 analyzes the Reassembled WSI and extracts a plurality of features from each of the identified nuclei and membrane image segments detected in each of the one or more ROIs in the Reassembled WSI. Depending on the tissue sample captured in the WSI, a plurality of features may be extracted by the Module 124 from detected nuclei and membrane segments in each of the one or more ROI in the Reassembled WSI.

The plurality of features extracted by the Module 124 from each of the ROI in the Reassembled WSI includes features associated with the membrane and nuclei detected by the CNN model 128 in each of the respective ROI that are relevant to HER2 scoring of the tissue sample (according to ASCO/CAP guidelines) and will be used to generate the one or more regional HER2 score predictions 108 for each of the one or more respective ROI in the Reassembled WSI.

As an example, the Extraction Module 124 may extract from the nuclei and membrane detected in each of the one or more ROI in the Reassembled WSI features including but not limited to: membrane stain intensity; underlying color of membrane—intensity of the underlying color of membrane; membrane stain deviation—standard deviation in the intensity detected throughout the captured image of the membrane; membrane stain completeness ratio—ratio of complete membrane stain to all membrane area detected; nuclei stain intensity; underlying color of nuclei—intensity of underlying color of nuclei; nuclei stain deviation—standard deviation in the intensity detected throughout the captured image of the nuclei; membrane skewness—skewness factor in the histogram curve of detected membrane; nuclei skewness—skewness factor in the histogram curve of detected nuclei; nuclei and membrane ratio—ratio of number of membrane detected by number of nuclei detected; membrane filled area—area of complete membrane staining based on area of detected nuclei; tumor area—tumor volume/area captured by image; cell percentage—percentage of completely stained membrane; and DAB mean color—mean RGB color value of DAB component of HED (Haematoxylin-Eosin-DAB) color space in image.

To analyze and extract the one or more plurality of features from the nuclei and membrane in each of the one or more ROI in the Reassembled WSI, the Processing and Feature Extraction Module 124 may include and use one or more of the following image processing libraries or tools, without limitation, to process the Reassembled WSI: OpenCV (CV2), Scikit-Image (skimage), SciPy, Pillow/PIL, NumPy, Mahotas, SimplelTK, Pgmagick, and the like.

Upon extracting one or more of the plurality of membrane and nuclei features from the ROI in the Reassembled WSI, the Module 124 further classifies each of the plurality of extracted features into one of a plurality of feature categories associated with ASCO/CAP guidelines for IHC HER2 scoring. As an example, a “membrane stain intensity” feature extracted from the membrane depicted in ROI of the Reassembled WSI may be further classified into one of a plurality of categories including “intensely stained,” “moderately stained,” or “not stained.” Other examples of classified extracted features include whether the image segment depicts a completely stained membrane, a partially stained membrane, a stained membrane that is skewed more towards the color brown, and/or a membrane that is skewed more towards the color white.

The one or more of the plurality of extracted and classified features of the nuclei and membrane in each of the ROI may then be outputted as WSI Extracted Data 106 to the HER2 Prediction System 126 for the generation of one or more regional HER2 score prediction 108 in accordance with ASCO/CAP guidelines.

At block 216, the HER2 Prediction System 126 determines if a trained second machine learning for scoring each of the ROI is available. In the embodiment described, the second machine learning model may be ML Model 132 described in FIG. 1. If a trained version of ML Model 132 is not available, the process proceeds to block 218 where ML Model 132 is trained with labeled training data. Otherwise, the process proceeds to block 220.

At block 220, the HER2 Prediction System 126 uses the trained ML Model 132 to classify each of the one or more ROI with one or more regional HER2 score predictions 108 using the WSI Extracted Data 106 from each respective ROI. In one aspect, the one or more regional HER2 score predictions 108 outputted by the trained ML Model 132 are based ASCO/CAP guidelines for IHC HER2 scoring. The ML Model 132 may be retrained in HER2 scoring to correspond with changes and updates to ASCO/CAP guidelines for IHC HER2 scoring of IHC stained cancer tissue samples.

Lastly, at block 222, the overall HER2 score prediction 109 for the WSI based on the one or more regional HER2 score predictions 108 is outputted to the User Interface 140. In the instance when the entire tissue sample in the WSI is designated as a single ROI, the overall HER2 score prediction 109 may be the same as the regional HER2 score prediction outputted by the trained ML Model 132 for the single designated ROI.

As an example, according to ASCO/CAP guidelines for IHC HER2 scoring, a region of IHC stained breast cancer cells is scored as IHC3+ if the stained membrane in the IHC stained tissue cells show complete circumferential membrane staining that is complete, intense, and in greater than 10% of tumor cells observed within a homogenous and contiguous invasive cell population. As such, if the WSI Extracted Data 106 analyzed at block 220 by the trained ML Model 132 for a region of stained tissue cell includes feature data classified as “membrane intensely stained,” “membrane completely stained,” and percentage or ratio of number of detected intensely and completely stained membrane to total number of nuclei detected in the respective region is greater than 10%, then the trained ML Model 132 would likely output a regional HER2 score of IHC3+ for the respective analyzed region of stained tissue cell.

Taking the total number of nuclei detected in the respective evaluated region as the total number of tumor cells observed in the region being scored, the percentage representing the ratio of the number of stained membrane to total nuclei detected in the evaluated region being greater than 10% indicates the number of tumor cells that had their membranes intensely and completely stained by IHC was in more than 10% of the tumor cells in the region.

As mentioned above, the image of the invasive stained tissue sample captured in the WSI may include multiple groups/regions of stained tissue cells or ROI. In an embodiment, one or more regional HER2 score predictions 108 corresponding to each of the one or more designated ROIs in the WSI would be outputted by the HER2 Prediction System 126. In an example where more than one regional HER2 score predictions 108 are generated for multiple ROIs in the WSI, the HER2 Prediction System 126 also outputs the overall HER2 score prediction 109 for the WSI as a whole based on the one or more regional HER2 score predictions 108 and ASCO/CAP guidelines.

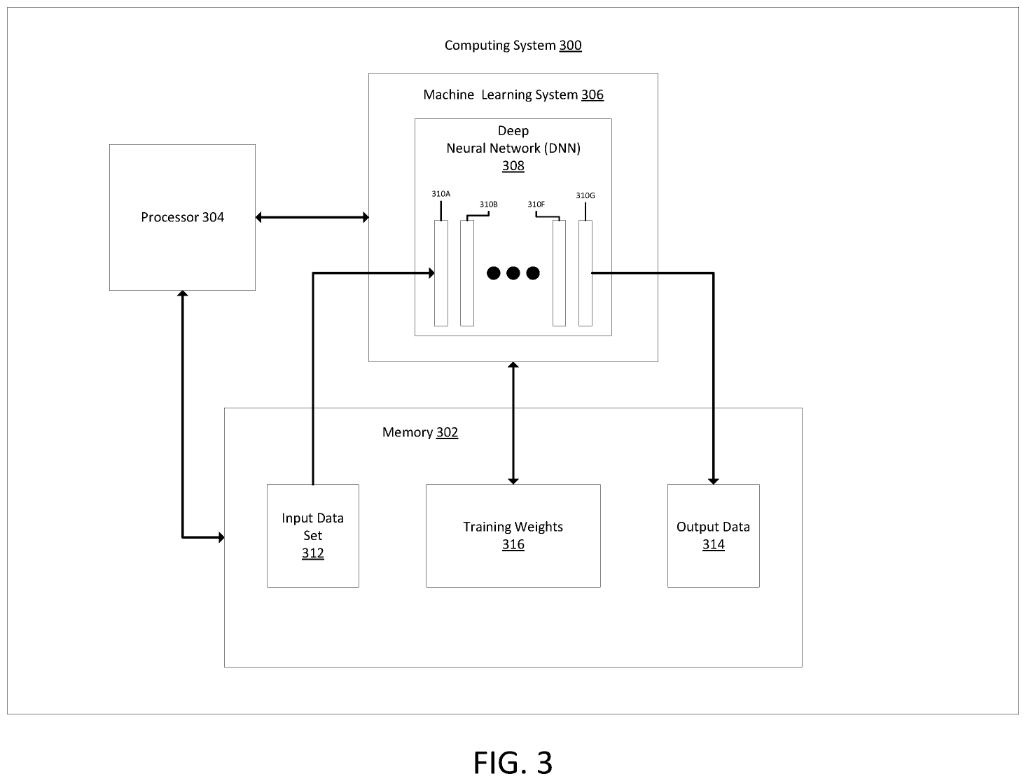

FIG. 3 illustrates an example of a deep-learning image analysis computing system 300 utilized by the Detection System 122 for detecting nuclei and membranes in WSI input data 102, according to certain embodiments.

Deep-learning is a subset of machine learning methods that are based on learning representations in data. Deep-learning uses a set of algorithms to model high-level abstractions in data using a deep graph with multiple processing layers including linear and non-linear transformations. The word ‘deep’ refers to layered/hierarchical learning. While many machine learning systems are seeded with initial features and/or network weights to be modified through learning and updating of the machine learning network, a deep-learning network trains itself to identify “good” features for analysis. A fundamental building block of a deep-learning neural network is a perceptron. The perceptron is an algorithm for supervised learning of binary classifiers that is composed of a linear component (weighted-sum of inputs) and a non-linear-component (activation). Combining perceptrons in multiple layers enables representation of complex features for addressing a multitude of real-world problems and discrepancies from substrate to substrate.

As previously discussed, ASCO/CAP guidelines require a minimum of 10% of cancer cells in a tissue sample to have been evaluated when assessing an IHC3+ or IHC2+ HER2 score for the respective tissue sample. The 10% requirement similarly applies to evaluating each of the one or more ROIs depicted in then WSI when assessing a regional HER2 score for the respective ROI of the scored tissue sample. Since each cancer cell depicted in the one or more ROIs of the WSI can be presumed to have a single nuclei, the total number of nuclei detected in the one or more ROIs of the WSI may therefore correspond with the total number of cancer cells in the WSI. Information concerning the number of nuclei in the one or more ROIs of the WSI may therefore be utilized to determine the percentage of cancer cells in the WSI as a whole a predicted regional HER2 score is based on for each of the one or more ROIs.

As an example, in order for a ROI in the WSI to receive an IHC1+, IHC2+, or IHC3+ HER2 score prediction, the number of cancer cells in the respective ROI the regional HER2 score prediction is generated for must contain more than 10% of the cancer cells in the WSI. That is, the number of detected nuclei in the ROI being scored must be greater than 10% of the total number of detected nuclei in all of the one or more ROIs in the WSI combined. As such, due to the 10% ASCO/CAP guideline requirements, ROIs that would have received a non NCO HER2 regional score prediction (e.g. based on classification of other extracted features such as membrane staining etc.) but contain a nuclei count less than 10%, would nonetheless be given an NCO score. Being able to quantify the number of cancer cells throughout the WSI may therefore ensure that regional and overall HER2 score predictions for the tissue sample depicted in the WSI meet the 10% ASCO/CAP guideline.

As shown in FIG. 3, in an embodiment, computing system 300 may include a memory 302, one or more processors 304, and processing circuitry for executing machine learning system 306 having a deep neural network (DNN) 308 made up of a plurality of layers 310A through 310G (collectively, “layers 310”). DNN 308 may be one of various types of deep neural networks (DNNs), such as convolutional neural networks (CNNs), as in CNN 128, feedforward neural networks, recursive neural networks (RNNs), and the like.

Memory 302 may store information for processing during operation of computing system 300. Memory 302 may store program instructions and/or data associated with one or more of the systems and modules described in accordance with one or more aspects of this disclosure. The one or more processors 304 may execute instructions and the one or more storage devices for memory 302 may store instructions and/or data of one or more modules. The combination of processors 304 and memory 302 may retrieve, store, and/or execute the instructions and/or data of one or more applications, modules, or software.

The one or more processors 304 and memory 302 may provide an operating environment or platform for one or more modules or units, which may be implemented as software, but may in some examples include any combination of hardware, firmware, and software. The one or more processors 304 and/or memory 302 may also be operably coupled to one or more other software and/or hardware components, including, but not limited to, one or more of the components and/or systems illustrated in FIG. 1 and other figures of this disclosure.

In the example of FIG. 3, DNN 308 having a convolutional neural network as in CNN 128 receives input data from input data set 312 and generates an output data 314. Input data set 312 and output data 314 may contain various types of information. For example, input data set 312 may include a plurality of image data associated with one or more IHC stained tissue samples. Output data 314 may include classification data, translated text data, and/or image classification data associated with the input data set 312. As disclosed in process 200 above, input data set 312 may correspond with captured WSI images of tissue sample as in WSI input data 102. As disclosed in process 200, output data 314 may correspond with classification data of the image data segments from input data set 312, as with the providing of WSI Detected Data 134 by the CNN model 128 in Detection System 122.

In an embodiment, DNN 308 may include a plurality of layers 310A through 310G (collectively, “layers 310”). Each of layers 310 may include a respective set of artificial neurons. Layers 310 include an input layer 310A, an output layer 310G, and one or more hidden layers (e.g., layers 310B through 310F). The output from the first layer 310A is fed into intermediate hidden convolution layers 3106 through 310F that analyze each of the usable image segments. The intermediate hidden layers 310B through 310F are fed into the final output layer 310G identifying and classifying whether an analyzed image segment from the WSI input data 1020 depicts an image of a nuclei, a membrane, or neither (background).

Layers 310 may include fully connected layers, convolutional layers, pooling layers, and/or other types of layers. In a fully connected layer, the output of each neuron of a previous layer forms an input of each neuron of the fully connected layer.

In a convolutional layer, each neuron of the convolutional layer processes input from neurons associated with the neuron's receptive field. Specifically, each of the convolution layers in layers 310 may include one or more filters. The filters can detect patterns in images and form layers within each convolutional layer. All neurons in a filter share the same weights and each neuron in one filter is connected to its counterpart in the next filter so that the output of one filter is passed to the corresponding set of neurons in the next filter.

The purpose of each filter is to detect different patterns in the image and as the DNN 308 is trained, it will converge or “learn” on which patterns (e.g. features related to nuclear/membrane morphology) within each filter will enable it to recognize each image. The output of each filter is called a feature map and it is these that are used by the DNN 308 to decompose an image down into its component pieces. Neurons in a filter evolve specific patterns and fire when they see that pattern and output the result into the next filter. All of these feature maps can then be build up into the final image and can be used to process it.

Pooling layers combine the outputs of neuron clusters at one layer into a single neuron in the next layer. Unlike convolution layers, pooling layers are not trainable. Their purpose are to sub-sample an image highlighting the most important areas for the network to process. Pooling layers are used to reduce computational complexity and to reduce the dimensionality of the image.

An example of a pooling layer is a “max-pooling layer.” Max-pooling layers look at all of the pixels in a receptive field, picks the one with the highest value (max value) and passes that on to the next layer in the network. Alternatively, other popular forms of pooling including but not limited to average-pooling may be used by the DNN 308.

Memory 302 stores a plurality of training weights 316 for DNN 308. Each input of each artificial neuron in each layer 310 of DNN 308 is associated with a corresponding training weight 316. Use of DNN 308 with the plurality of training weights 316 yields output data 314.

As described in further detail below, as part of performing the training process, machine learning system 306 may perform a feed-forward phase in which machine learning system 306 uses the plurality of training weights 316 in DNN 308 to determine output data 314 based on input data in input data set 312. Furthermore, machine learning system 306 may perform a backpropagation method that calculates a gradient of a loss function. The loss function produces a cost value based on the output data. Machine learning system 306 may then update the plurality of training weights 316 based on the gradient of the loss function. Machine learning system 306 may perform the feed-forward method and backpropagation method many times with different input data. During or after completion of the training process, machine learning system 306 may use trained weights in an evaluative inference process to generate output data 314 based on new non-training input data.

In another aspect of the embodiment, as mentioned above, the neurons in a filter of a convolution layer all share the same weightings. As such, the machine learning system 306 may utilize shared training weights 316 for classifying both nuclei and membrane detected in analyzed image segment inputs. Use of shared training weights 316 allow for the detecting of both nuclei and membrane by the machine learning system 306 without additional computation. For example, when compared to the use of parallel binary classifiers to analyze each image segment input to detect membrane and nuclei separately, utilization of shared training weights 316 by the machine learning system 306 is a technical improvement in computational efficiency in allowing for multiple structures to be detected without added computation. Furthermore, since the weights of the filters are tied, this means the filters do not change as the filter analyzes each portion of the whole slide image. This shared weightings means that once the filter has learned to recognize a particular shape or feature in an image, the network will recognize the specific shape or feature regardless of the whereabouts of the shape or feature in the image since the same filter is being used. Use of the shared weights reduces the amount of weights (or parameters) that need to be optimized when training the machine learning system 306. Fewer calculations also indicate that the machine learning system 306 can be trained both faster, and often with less data.

The deep-learning neural network 308 trained and deployed as discussed herein may, in some aspects, include machine learning models trained to recognize spatial relationships between pixel data in an input data set for which the machine learning models are to make a determination or classification. For example, the deep-learning model may include other multi-layer neural network-based models in which pixel information and the relationships between different items in a spatial sequence can be learned, including but not limited to, single shot detection (SSD), region based convolutional neural network (R-CNN), Faster region based convolutional neural networks (Faster R-CNN) and You Only Look Once (YOLO).

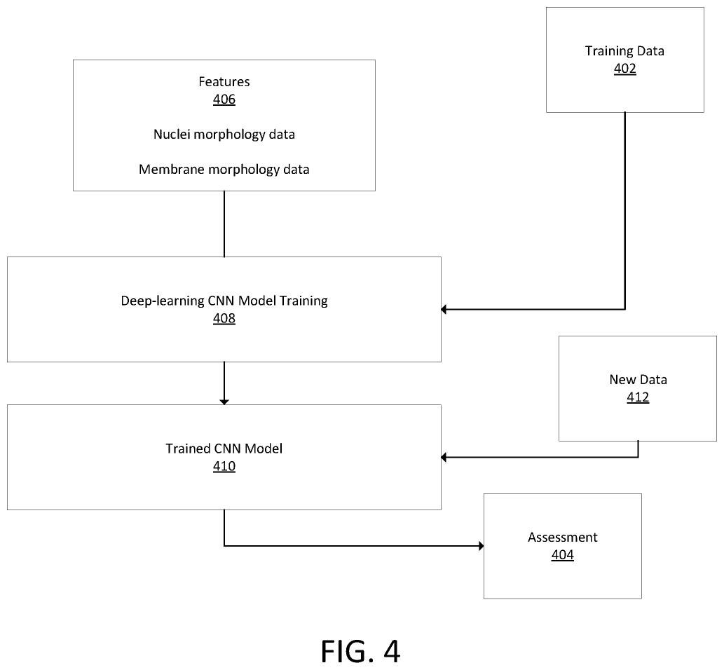

FIG. 4 depicts a schematic diagram showing an example of the training and use of a deep-learning machine learning, as in CNN 128, to detect nuclei and membrane in input images of stained tissue cells, according to certain embodiments. As discussed in this disclosure, deep-learning is a subset of machine learning based on learning representations in data. Machine learning, including deep-learning, explores the study and construction of algorithms, also referred to herein as tools, which may learn from existing data and make predictions about new data. In an embodiment, such tools operate to build and train a trained deep-learning CNN Model 410 from example training data 402 in order to make data-driven predictions or decisions expressed as outputs or assessments 404. Although example embodiments are presented with respect to a few machine-learning tools, the principles presented herein may be applied to other machine-learning tools.

In the embodiment shown, the training of the deep-learning CNN model includes utilizing one or more features 406 for analyzing the training data 402 to generate one or more assessments 404. A feature 406 is an individual measurable property of a phenomenon being observed. The concept of feature is related to that of an explanatory variable used in statistical techniques such as linear regression. In one example embodiment, the features 406 utilized to detect membrane and nuclei in segments of images may include one or more features associated with nuclei morphology and membrane morphology. In another aspect, training data 402 for features 406 may be of different types and may include one or more of image data, colors, size, shape, position, brightness, and the like. One of the many advantages of deep-learning includes automatic extraction of features for the classification problem at hand, as opposed to engineering hand-crafted features. As such, the CNN model utilizes the training data 402 to distinguish and learn meaningful features 406 that are useful in classifying the one image or images or portions of images from the WSI input data 102 to produce the outcome or assessment 404.

In some example embodiments, to train the CNN model to detect nuclei and membrane in the WSI input data 102, the training data 402 may be reviewed and labeled before training to provide structured and supervised learning when training the deep-learning CNN model. As such, in an embodiment, the training data 402 may include labeled tissue cell image segments of nuclei and membrane. In addition to labeling the training data 402, the training data 402 may also be further prepared and augmented to more robustly train the CNN model to be scanner-agnostic.

In an embodiment, images from image data source 110 may vary due to different imaging systems used to capture the WSI training data 402. The images in the WSI training data 402 may also vary due to varying protocols, procedures, and labs used for preparing and staining the tissue sample prior to viewing. In order to train the deep-learning CNN model to be more scanner agnostic, image segments used as training data 402 may be further modified to train the CNN model to detect images of nuclei and membrane of varying sizes, colors, intensities, positions, and orientations. For example, prior to training the CNN model, the training data 402 may be modified and augmented, without limitation, with varying contrast, brightness, blurriness, sharpness, cropping, cutting and mixing, elastic transformation and colors to accommodate for different types of scanners. The tissue sample slides used to generate the image segments for the training data 402 may also be captured in varying rotation and orientations to accommodate for varying positions and orientations of the slides during the capturing of images of the stained tissue sample to be scored. Such training of the CNN model may allow the system 100 to be more robust, scanner agonistic, and capable to analyze and generate HER2 score predictions for images of stained tissue samples of varying qualities, settings, and properties.

With the training data 402 and the learned features 406, the CNN model is trained at operation 408 to result in the trained CNN Model 410. When the trained CNN Model 410 is used to perform an assessment, new data 412 is provided as an input to the trained CNN Model 410, and the CNN Model 410 generates the assessment 404 as output based on the features 406 learned by the CNN Model 410. For example, when future WSI of stained tissue samples are analyzed by the trained CNN Model 410, the CNN Model 410 utilizes features 406 it learned about the morphology of membrane and nuclei to detect each of the nuclei and membrane in the image from new data 412. Another advantage of deep-learning is the ability to perform transfer learning, i.e., once the model has been trained on a large dataset, there is not a need to train the model again from scratch for a new dataset.

As such, referring to the system 100 in FIG. 1, once the CNN Model 128 is trained in the same manner as trained CNN Model 410 discussed above, the CNN Model 128 is used by detection system 122 to detect and identify segments of nuclei and membrane in the input image obtained from Image Data Source 110.

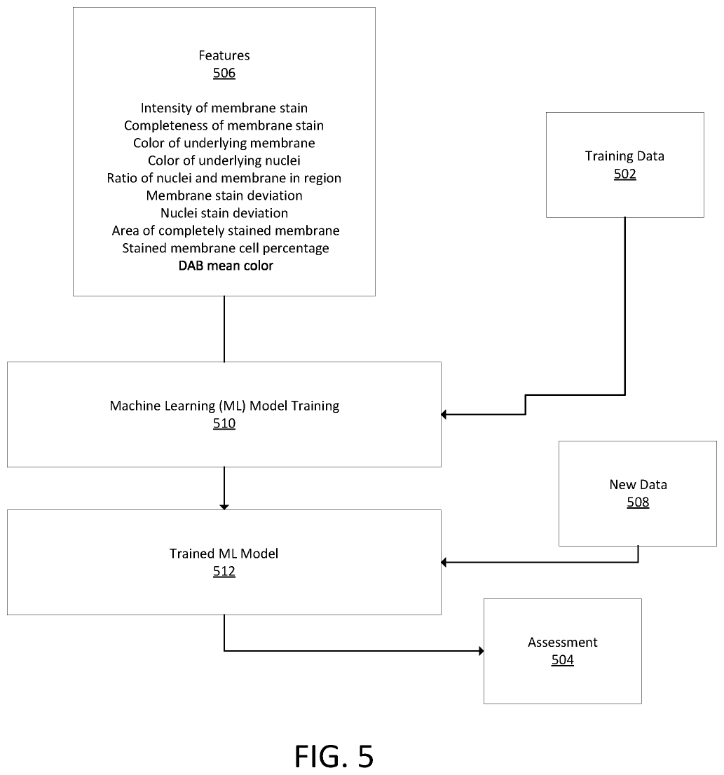

FIG. 5 depicts a schematic diagram showing an example of the training and use of a machine learning model to classify a regional HER2 score prediction for one or more ROI in a Reassembled WSI, according to certain embodiments.

In the embodiment shown, the machine learning model may utilize one or more features 506 for analyzing the training data 502 to generate assessments 504. In one example embodiment, the features 506 utilized to classify one or more regional HER2 score prediction 108 may include the plurality of membrane and nuclei feature classification data of an image segment previously detected by the Detection System 122 and classified by the Processing and Feature Extraction Module 124. For example, data related to features 506 in an ROI image segment used by the machine learning model to generate assessments 504 may include, without limitation, classification data corresponding to the following plurality of membrane and nuclei features: membrane stain intensity; underlying color of membrane—intensity of the underlying color of membrane; membrane stain deviation—standard deviation in the intensity detected throughout the captured image of the membrane; membrane stain completeness ratio—ratio of complete membrane stain to all membrane area detected; nuclei stain intensity; underlying color of nuclei—intensity of underlying color of nuclei; nuclei stain deviation—standard deviation in the intensity detected throughout the captured image of the nuclei; membrane skewness—skewness factor in the histogram curve of detected membrane; nuclei skewness—skewness factor in the histogram curve of detected nuclei; nuclei and membrane ratio—ratio of number of membrane detected by number of nuclei detected; membrane filled area—area of complete membrane staining based on area of detected nuclei; tumor area—tumor volume/area captured by image; cell percentage—percentage of cell completing membrane, and DAB mean color—mean RGB color value of DAB component of HED (Haematoxylin-Eosin-DAB) color space in image.

In an embodiment, classification data corresponding to one or more of the above plurality of nuclei and membrane features in a plurality of image segments of nuclei and membrane may be used as training data 502 to provide structured and supervised learning to train the machine learning model to classify each of the one or more ROI in the WSI input data 102 with a regional HER2 score prediction 108 ranging from NCO to IHC3+.

The machine learning model utilizes the training data 502 to find correlations among the identified features 506 that affect the outcome or assessment 504. In some example embodiments, the training data 502 includes known labeled data for one or more plurality of identified features 506 for respective nuclei and membrane detected in a plurality of image segment and one or more outcomes/classifications, including the corresponding HER2 score prediction assessment based on ASCO/CAP guidelines for the captured and classified image of stained nuclei and membrane cell used in training data 502.

With the training data 502 and the identified features 506, the machine learning model is trained at operation 510. The result of the training is the trained ML Model 512. The filters for the trained ML Model 512 extract the identified features 506 from future images as a feature map to get an optimal classification performance.

According to certain embodiments, ML Model 512 may include one or more of a random forest walk, a support vector machine, a decision tree, a convolutional neural network, or other machine learning model capable of classifying images, or portions of images, based on being trained by the training data 502. These machine learning models may, for example, be models capable of segmenting an image into a plurality of categories according to a categorization assigned to one or more of the plurality of features 506 in an ROI image segment (e.g. intense and complete membrane staining, weak and complete membrane staining, weak and incomplete membrane staining, and no staining). In segmenting an image into a plurality of categories, the machine learning models can generate a segmentation map dividing the image into a plurality of segments. Each segment of the plurality of segments may be associated with one of a plurality of features and categories with which the machine learning model was trained. Thus, the segmentation map can identify segments of an image as segments classified as intense and complete membrane staining, segments depicting weak and complete membrane staining, non-stained membrane patterns, and other types of membrane staining patterns and generate a corresponding regional HER2 score prediction based on the classification of the plurality of features and categories for the respective image segment.

When the ML Model 512 is used to perform an assessment, new data 508 is provided as an input to the trained ML Model 512, and the ML Model 512 generates the assessment 504 (a regional HER2 score prediction) as output. For example, when future captured and classified tissue cell images of stained nuclei and membrane are analyzed by the trained ML Model 512, the ML Model 512 utilizes the classification data of one or more of the plurality of features 506 of the nuclei and membrane in the image of new data 508 to output one or more HER2 score predictions in assessment 504 for the respective analyzed ROI in the image of new data 508.

In another aspect of the present disclosure, the algorithm parameters corresponding to the training of the ML Model 512 may be further adjusted and calibrated after initial use by the user to further train the ML Model 512 to be more robust in accurately generating a HER2 score prediction for new stain and image variations not previously considered during the training of the ML Model 512.

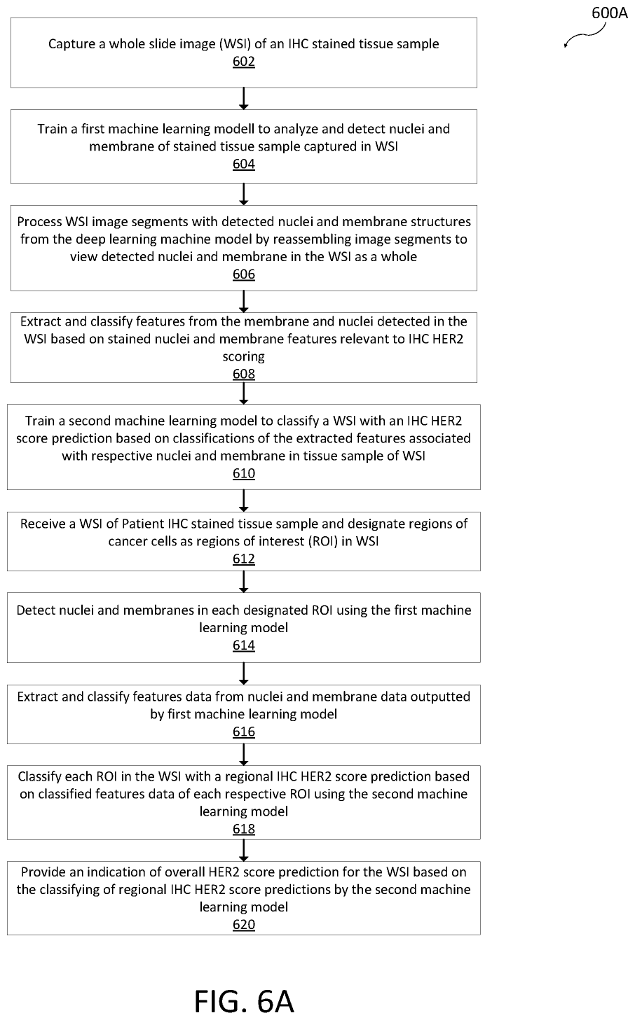

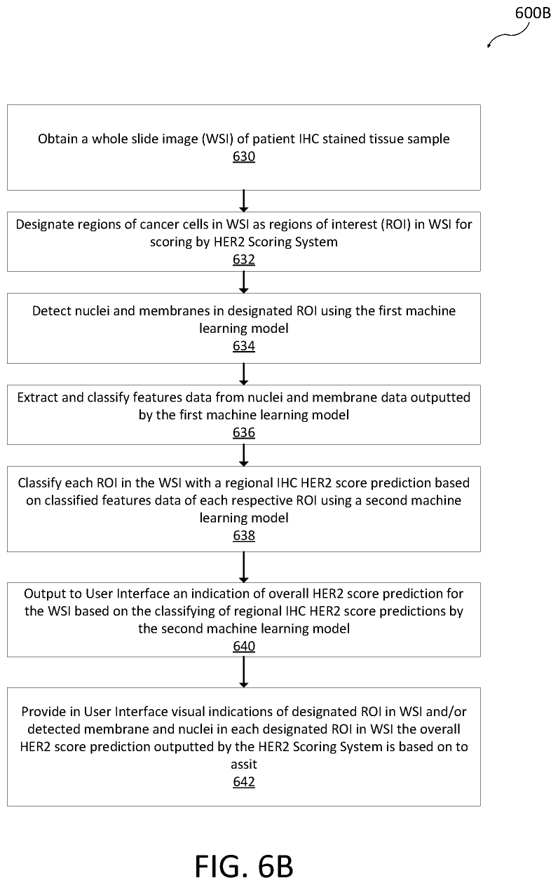

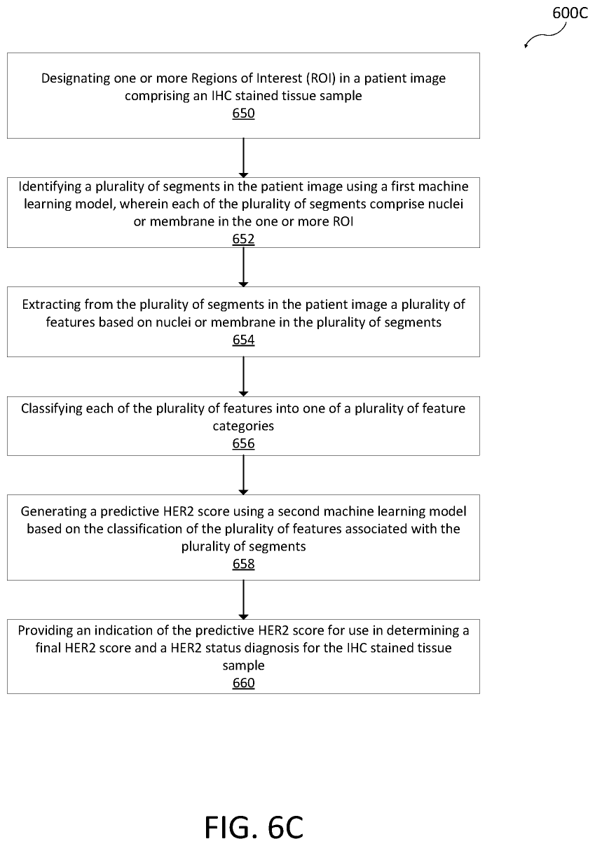

FIG. 6A depicts a flow diagram of an example method 600A for training and use of the system in FIG. 1 with the processes of FIGS. 2, 4, and 5, according to certain embodiments.

At block 602, a WSI of an IHC stained tissue sample is obtained by viewing the IHC stained tissue sample through a microscope and capturing an image of the microscopic structures of the stained tissue sample.

At block 604, a first machine learning model is trained to detect and identify nuclei and membrane in images of stained tissue samples captured in the WSI. According to certain embodiments, the first machine learning model is a deep-learning model having a convolutional neural network as in CNN Model 128 in the Detection System 122. In one aspect, the first machine learning model analyzes the WSI in 512×512 pixel image segments and outputs respective classified 512×512 image segments. Image segments outputted from the first machine learning model may be classified as (1) “nuclei” if nuclei are detected in the respective image segment, (2) “membrane” if membrane are detected in the respective image segment, or (3) skipped if neither nuclei nor membrane are detected.

At block 606, the classified nuclei and membrane image segments from the first machine learning model are processed by being reassembled and stitched together to allow for the detected nuclei and membrane in the WSI to be viewed and analyzed as a whole in the context of the entire stained tissue sample captured in the original WSI. At block 608, features relevant to IHC HER2 scoring and associated with the detected membrane and nuclei in the reassembled WSI image are extracted and classified based on features relevant to IHC HER2 scoring guidelines.

At block 610, a second machine learning model is trained to analyze and classify regions of cells in the WSI with regional IHC HER2 score predictions based on the classified extracted features of the stained nuclei and membrane detected in the cells in each respective region of the WSI. According to certain embodiments, the second machine learning model may be one of a random forest machine learning, as in ML Model 132 in the HER2 Prediction System 126, support vector machine, a decision tree model, or any other machine learning model capable of learning and solving classification problems.

At block 612, a WSI of a patient IHC stained tissue sample is received and one or more regions of interest (e.g. stained cancer cells) are designated for scoring. At block 614, a plurality of segments is outputted by the first machine learning wherein each of the plurality of segments corresponds to nuclei and/or membranes detected in each of the one or more regions of interest (ROIs) designated in the WSI. At block 616, the plurality of nuclei and membrane image segment data outputted by the trained first machine learning model is processed and reassembled together to correspond to the original WSI input. Data associated with a plurality of features of the nuclei and membrane detected by the trained first machine learning model in the one or more ROIs of the WSI are then extracted and classified in one of a plurality of categories associated with the plurality of features in accordance with ASCO/CAP guidelines for IHC HER2 scoring.

At block 618, each of the one or more ROI corresponding to each homogenous and contiguous invasive stained cell population depicted in the reassembled WSI is classified with a regional HER2 score prediction using the trained second machine learning model based on the classification assigned to the plurality of extracted features associated with the nuclei and membrane detected by the trained first machine learning model in each of the respective ROI in the WSI. During the scoring of each of the one or more ROI by the trained second machine learning model, one or more intermediate outputs may be outputted to the User Interface Visualization Output 140 to provide details and data regarding how the one or more regional HER2 score predications are being generated.

In one aspect of the embodiment, in block 618 where each ROI depicted in the reassembled WSI is classified with a regional HER2 score prediction, the regional HER2 score predictions are generated based on the classification assigned to the plurality of extracted features associated with the nuclei and membrane detected by the trained first machine learning model in each of the respective ROI in the WSI. Due to the extracted features classified being associated with and based on both nuclei and membrane detected by the trained first machine learning model (with regions where only membrane detected being discarded and not utilized in generating HER2 score predictions), the method 600 therefore generates more accurate HER2 score predictions than conventional methods that rely exclusively on stained membrane detection.

Lastly, at block 620, the overall HER2 score prediction is 109 outputted and provided for reference by a user in assessing a final HER2 score for the WSI and status diagnosis for the patient IHC stained tissue sample obtained in block 612. In the instance when more than one regional HER2 score predictions 108 are generated for a single WSI based on more than one ROI being designated in the WSI, the overall HER2 score prediction 109 is outputted by the HER2 scoring system 120 based on the one or more regional HER2 score predictions 108 generated for each of the one or more designated ROIs. In an embodiment, the overall HER2 score prediction outputted for the WSI by the HER2 scoring system 120 corresponds to the highest regional HER2 score prediction 108 generated by the HER2 scoring system 120 based on the one or more ROIs analyzed and scored.

FIG. 6B depicts a flow diagram of an example method 600B for use of the system in FIG. 1 to generate a predictive HER2 score for a WSI of a patient IHC stained breast cancer tissue sample, according to certain embodiments.

At block 630, the WSI of the patient IHC stained tissue sample is obtained from image data source 110 for analysis and scoring by the HER2 scoring system 120. Obtaining the patient WSI from image data source 110 may include viewing and capturing via a digital camera and/or a digital microscope a prepared stained slide specimen by the user. Alternatively, obtaining the patient WSI from image data source 110 may include uploading the patient WSI from a third party source and/or external database.

In block 632, one or more regions of stained cancer cells in the WSI are designated as regions of interest (ROI) in the WSI for scoring by the HER2 scoring system 120. At block 634, each of the ROIs in the WSI are analyzed by a first machine learning and a plurality of segments is outputted by the first machine learning wherein each of the plurality of segments corresponds to nuclei and/or membranes detected in each of the one or more ROIs designated in the WSI. At block 636, features data are extracted from the plurality of nuclei and membrane image segment data outputted by the trained first machine learning model and classified for IHC HER2 scoring. To extract the features data, the plurality of nuclei and membrane image segment data are processed and reassembled together in a reassembled WSI corresponding to the original WSI input for assessment of the extracted features data by corresponding designated ROIs. The extracted features data are based on a plurality of features corresponding to the nuclei and membrane detected by the trained first machine learning model in each of the one or more ROIs and are classified in one of a plurality of categories in accordance with ASCO/CAP guidelines for IHC HER2 scoring.

At block 638, each of the one or more ROIs depicted in the reassembled WSI are then classified with a regional HER2 score prediction 108 using a second machine learning model based on the classification assigned to the plurality of extracted features associated in block 636. During the scoring of each of the one or more ROI by the second machine learning model, one or more intermediate outputs may be outputted to the User Interface Visualization Output to provide details and data regarding how the one or more regional HER2 score predications are being generated.

In an embodiment, when the second machine learning model is determining between NCO and IHC1+ as the regional HER2 score prediction 108 of any of the one or more ROIs in the reassembled WSI, the second machine learning model may use the DAB mean color of the respective ROI to finally determine between NCO and IHC1+. The DAB mean color of the one or more ROIs refers to the DAB component in the HED (Haematoxylin-Eosin-DAB) color space of the detected membranes for assessing the “brownness” of the ROI in the image. In some embodiments, the DAB mean color includes a RGB value range between 0-255.

The DAB mean color of each of the ROI may be calculated by first taking the original RGB image of membranes detected in the ROI by the first machine learning model and applying a mask that converts/indicates the color/brownness of the space in the image under the membrane. The HED color space component for the ROI may then be calculated for the masked image and the DAB channel or component of the HED extracted with the other two components (Haematoxylin and Eosin) zeroed out. Upon extraction of the DAB HED component, the image is converted back to RGB color space and a mean of the DAB component corresponding to the ROI is obtained. It is appreciated that a threshold of the RGB value corresponding to the DAB mean color of the ROI may be used by the second machine learning model to finally decide between classifying the ROI as NCO and IHC1+, which in turn can affect the overall HER2 score prediction 109 of the WSI for determining the status diagnosis of the corresponding sampled tissue. For example, the threshold utilized by the second machine learning model can be 189 for the DAB mean color of the ROI. For ROIs in which the second machine learning model is to determine between NCO and IHC1+, the second machine learning model classifies the ROI as IHC1+ if the DAB mean color for the respective ROI is less than 189, and IHC0 if the DAB mean color is greater than 189.

At block 640, the overall HER2 score prediction 109 is outputted to the User Interface 140 for the WSI based on the one or more regional HER2 score predictions 108 and provided for reference by the user in assessing a final HER2 score for the WSI and status diagnosis for the patient IHC stained tissue sample obtained in block 612. In the instance when more than one regional HER2 score predictions 108 are generated for a single WSI based on more than one ROI being designated in the WSI, the overall HER2 score prediction 109 is outputted by the HER2 scoring system 120 based on the one or more regional HER2 score predictions generated for each of the one or more designated ROIs. In an embodiment, the overall HER2 score prediction 109 outputted for the WSI by the HER2 scoring system 120 corresponds to the highest regional HER2 score prediction 108 generated based on the one or more ROIs analyzed and scored by the HER2 scoring system 120.

Lastly, in block 642, to assist the user in assessing a final HER2 score for the WSI and provide the user supporting data for the overall HER2 score prediction 109 generated by the HER2 Scoring System 120 to allow the user to review and confirm the prediction 109, the User Interface 140 may provide referencing visual indications on the WSI to provide support for and show how the prediction 109 was generated. The visual indications on the User Interface 140 may include overlaying the original obtained WSI with masks indicating the designated ROIs in the WSI and each of the corresponding regional HER2 score predictions 108 generated for each of the ROI. To provide the user further support regarding how each of the regional HER2 score predictions 108 were generated by the HER2 scoring system 120 for each of the designated ROI, the User Interface 140 may further overlay the original WSI with another mask identifying each of the nuclei and membrane detected by the first machine learning model for each of the designated ROIs in the WSI. The identifications of the nuclei and membrane for each ROI in the User Interface 140 also provides for review by the user corresponding features data that were extracted, classified, and used to generate regional HER2 score predictions 108.

FIG. 6C depicts a flow diagram of an example method 600C for use of the system in FIG. 1 to generate a predictive HER2 score, according to certain embodiments.

At block 650, one or more regions of stained cells are designated as regions of interest (ROI) in a patient image 700 comprising IHC stained tissue samples for scoring by the HER2 scoring system 120. At block 652, each of the ROIs in the WSI are analyzed by a first machine learning to identify a plurality of segments wherein each of the plurality of segments comprise nuclei or membrane in the one or more ROIs.

At block 654, a plurality of features are extracted from the plurality of segments outputted by the first machine learning model. In block 656, the plurality of features extracted from the plurality of segments are classified into one of a plurality of feature categories.

At block 658, a predictive HER2 score 702 is generated using a second machine learning model based on the classification assigned to the plurality of features extracted in block 654.

At block 660, an indication of the predictive HER2 score 702 is provided for reference by a user in determining a final HER2 score 704 and a HER2 status diagnosis 706 for the IHC stained tissue sample.

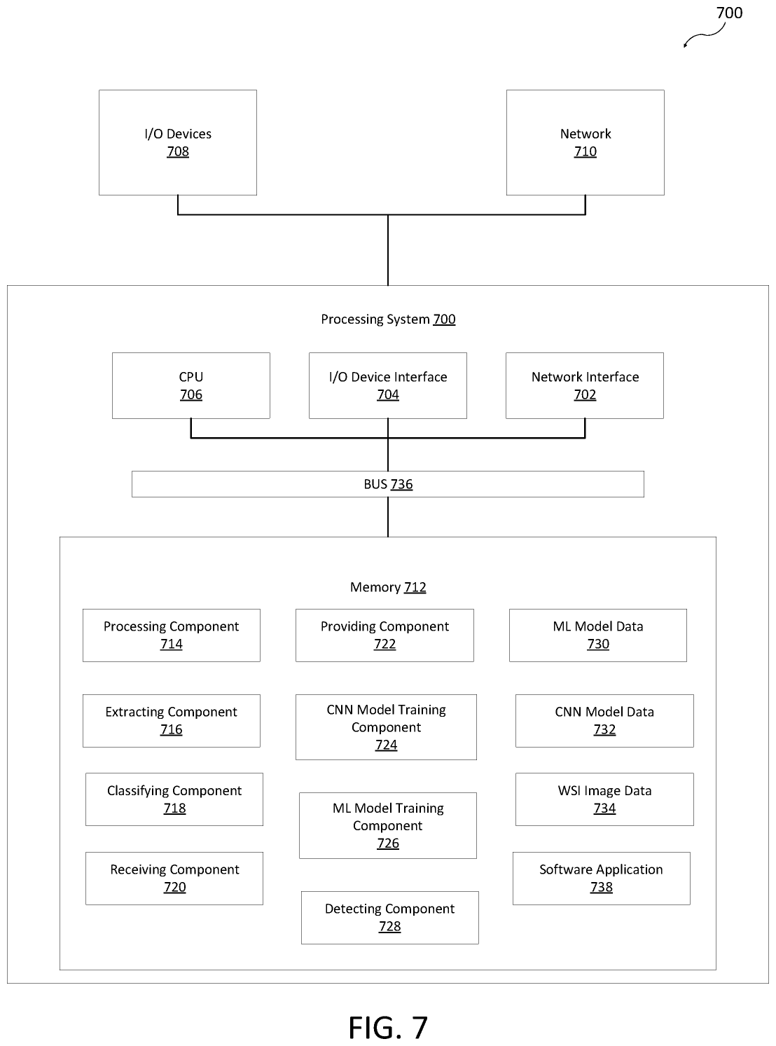

FIG. 7 depicts an example processing system 700, according to certain embodiments that may perform processes and methods described herein, such as the process for IHC HER2 scoring with respect to FIG. 2 and the method for HER2 score classification with respect to FIGS. 6A and 6B.

Processing system 700 includes a central processing unit (CPU) 706 connected to a data bus 736. CPU 706 is configured to process computer-executable instructions, e.g., stored in memory 712, and to cause the server to perform methods described herein, for example, with respect to FIG. 2. CPU 706 is included to be representative of a single CPU, multiple CPUs, a single CPU having multiple processing cores, and other forms of processing architecture capable of executing computer-executable instructions. Memory 712 is included to be representative of one or more memory devices such as volatile memories, that may be a RAM, cache, or other short-term memory that may be implemented in hardware or emulated in software, one or more non-volatile memories such as a hard drive, solid state drive, or other long term memory that may be implemented in hardware or emulated in software, or a combination of volatile and non-volatile memories. Moreover, one or more memory devices that makeup memory 712 may be located remotely from processing system 700 and accessed via a network.

Processing system 700 further includes input/output (I/O) device(s) 708 and interfaces 704, which allow processing system 700 to interface with input/output devices 708, such as, for example, keyboards, displays, mouse devices, pen input, and other devices that allow for interaction with processing system 700. Note that processing system 700 may connect with external I/O devices through physical and wireless connections (e.g., an external display device).

Processing system 700 further includes a network interface 702, which provides processing system 700 with access to external network 710 and thereby external computing devices.

Processing system 700 further includes memory 712, which in this example includes a processing component 714, an extracting component 716, a classifying component 718, a receiving component 720, a providing component 722, a CNN Model Training Component 724, a ML Model Training Component 726, and a Detecting Component 728, that may be used in performing operations described in FIGS. 2 and 6. Memory 712 further includes in this example ML Model Data 730, CNN Model Data 732, WSI Image Data 734, and one or more software applications 738 that may be used in performing operations described in FIGS. 2 and 6.

Processing system 700 can include one or more software applications 736 and media data stored by the memory 712 that is used by the CPU 706 to perform process 200 and the method 600 described herein. In some configurations, the CPU 706 includes a digital signal processor (DSP), an application-specific integrated circuit (ASIC), and/or a combination of such units. The CPU 706 is configured to execute the one or more software applications 736 and process the stored media data, which can be each included within the memory 712. The processing system 700 controls the transfer of data and files to and from the various systems and devices described in FIG. 1. The memory 712 is also configured to store instructions corresponding to any operation of the method 600 according to embodiments described herein.

Note that while shown as a single memory 712 in FIG. 7 for simplicity, the various aspects stored in memory 712 may be stored in different physical memories, including memories remote from processing system 700, but all accessible by CPU 706 via internal data connections such as bus 736.

Embodiments of the present disclosure may be provided to end users through a cloud computing infrastructure. Cloud computing refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g., an amount of storage space consumed by a user or a number of virtualized systems instantiated by the user). A user can access any of the resources that reside in the cloud at any time, and from anywhere across the Internet. In context of the present disclosure, a user may access software routines (e.g., one or more software applications 738 corresponding to HER2 Scoring System 120 to perform process 200 and the methods 600A and 600B) or related data available in the cloud. For example, the software routines could execute on a computing system in the cloud. In such a case, the software routines could maintain spatial and non-spatial data at a storage location in the cloud. Doing so allows a user to access this information from any computing system attached to a network connected to the cloud (e.g., the Internet).

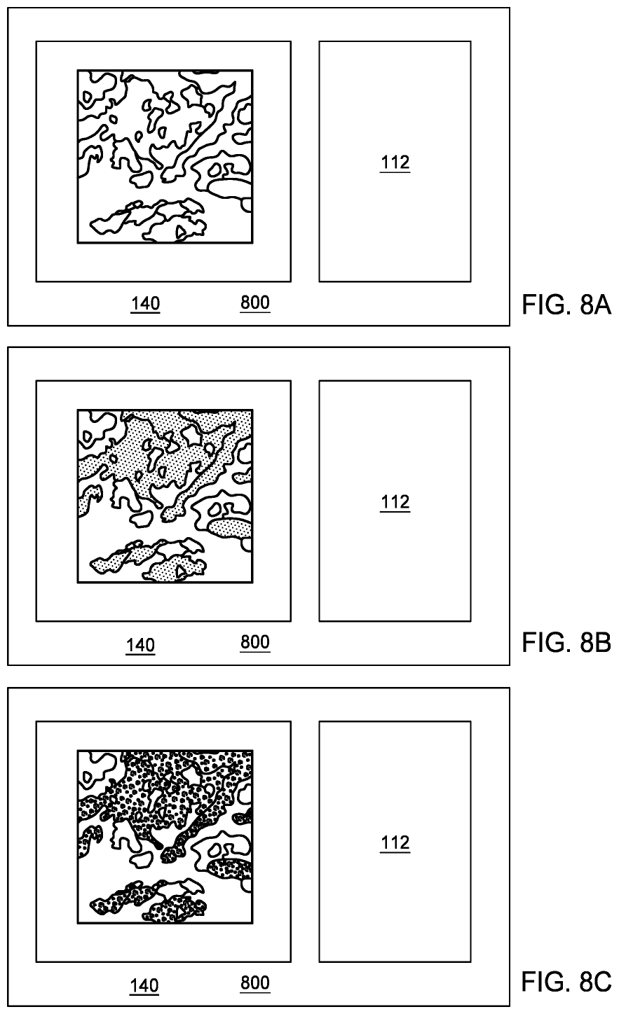

FIGS. 8A-8C depict an example User Interface Visualization Output 140, according to certain embodiments. Once the one or more regional HER2 score predictions 108 are obtained for each of the one or more ROI in the whole slide image of the IHC stained tissue sample, the HER2 scoring system 120 outputs the one or more regional HER2 score predictions 108 and corresponding analyzed whole slide image to the result visualization module 130. The result visualization module 130 then further processes the analyzed whole slide image and corresponding algorithm results related to the WSI detected data 104 and WSI extracted data 106, for output to the User Interface Visualization Output 140. Specifically, in an embodiment, due to the size of the whole slide image, the result visualization module 130 may process and prepare portions of the whole slide image at a time for viewing in the User Interface Visualization Output 140. The User Interface Visualization Output 140 may allow the user to navigate and alter the field of view in the User Interface 140 to view specific portions or ROIs of the WSI analyzed by the HER2 scoring system 120 for confirmation and reference with corresponding regional HER2 score predictions 108 generated by the HER2 scoring system 120.

In the User Interface Visualization Output 140, a prediction summary 112 is also prepared with the one or more regional HER2 score predictions 108 outputted for each of the respective ROIs, details for each of the one or more ROIs designated in the WSI and analyzed by the HER2 scoring system 120, the supporting related data each of the one or more regional HER2 score prediction 108 is based on for each of the analyzed ROI, and an overall HER2 score prediction for the tissue sample in the WSI if more than one regions of interest in the WSI was scored. FIG. 8A shows an example of the User Interface Visualization Output 140 with the prediction summary 112 accompanying an image of the original WSI analyzed by the HER2 scoring system.

In one embodiment, the prediction summary 112 includes information for each of the one or more ROIs designated in the WSI corresponding to a % nuclei positivity. The % nuclei positivity refers to the percentage of nuclei in the ROI for tissue samples captured in the WSI determined to be HER2 positive. The % nuclei positivity for each of the one or more ROIs in the WSI can be determined by dividing the number of nuclei detected in the respective ROI by the total number of nuclei detected in the WSI.

In some embodiments, information related to the % nuclei positivity of the one or more ROI can separately be based on completeness of membrane detected in the ROI of the WSI. For example, the % nuclei positivity in the ROI can be based on completeness of membrane dividable into four (4) categories, including where complete membrane is detected, no membrane is detected, both complete and incomplete membrane are detected, and only incomplete membrane detected. Calculating the % nuclei positivity for each of these categories can include dilating the nuclei in the image to determine the total number of detected nuclei in the WSI and using the dilated image as a mask to quantify the membrane instances in the WSI. Next, the quantified membrane instances can be used to calculate the pixels in each membrane instance with respect to the instances of each nuclei to determine the ratios of membrane to nuclei for each instance and binning the pixel/ratio data for each membrane into the four above-mentioned membrane completeness categories to determine the % nuclei positivity based on each.

The User Interface Visualization Output 140 further includes the option of overlaying the WSI image, shown in FIG. 8A, with a region mask 114 and a heat mask 116. The region mask 114 shows each of the ROIs designated and analyzed by the HER2 scoring system 120 for generating one or more region HER2 score predictions 108. The heat mask 116 shows each of the nuclei and membrane detected by the CNN model 128 for each respective ROI and utilized by the HER2 scoring system 120 for generating HER2 score predictions. FIG. 8B shows an example of the region mask 114 overlaid on the WSI with each of the ROI designated for reference by the user. In one aspect, the user may alternatively alter the ROI designations in the WSI through the User Interface Visualization Output 140 and generate new HER2 score predictions based on the new ROI designations.

FIG. 8C shows an example of the heat mask 116 generated and overlaid on each of the respective ROIs in the WSI designated in FIG. 8B. The heat mask 116 shows each of the detected nuclei and membrane in different colors or markings for comparison in each of the designated ROI. An image of the original WSI of the stained tissue sample may also be outputted to the user interface 140 for visually cross-referencing with the region mask 114 and/or the heat mask 116. As previously mentioned, given that ROIs that do not meet the 10% ASCO/CAP guideline are disregarded by the HER2 scoring system 120 for purposes of generating HER2 scoring predictions, the heat mask 116 allows the user to further review any ROIs that may have been disregarded to confirm the action by the HER2 scoring system 120 was proper. In one aspect, the heat mask 116 identifying and showing the nuclei and membrane detected by the HER2 scoring system 120 in the respective ROI may assist the user in confirming whether the disregarded ROI indeed did not meet the 10% ASCO/CAP guideline.

In another aspect of the embodiment, an overlaid view of the heat mask 116 on the WSI may allow the user track the nuclei and membrane in each of the ROIs in the WSI, while still being able to view the original color and staining of the various underlying microscopic components of the tissue sample as captured in the WSI. This may further allow the user to confirm whether the detected nuclei and membrane in the WSI by the HER2 scoring system 120 is accurate.

The prediction summary 112 may include information related to the number of detected nuclei and membrane in each of the ROIs in the WSI, as well as the respective regional HER2 score predictions 108 for each of the ROIs. Additional information may be provided regarding the extracted features of the membrane and nuclei detected in each of the ROIs used in classifying the one or more regional HER2 score predictions 108. An overall HER2 score prediction for the entire WSI may also be provided based on the one or more regional HER2 score predictions 108 outputted by the HER2 scoring system 120. The number of detected nuclei and membrane may be used to ensure that each of the regional HER2 score predictions 108 meet the 10% requirement in ASCO/CAP guidelines for IHC HER2 scoring of breast cancer tissue cells.

In another aspect, after the prediction summary 112 for the WSI is outputted by the HER2 Prediction System 126, a user may optionally adjust the ROIs analyzed by the HER2 Prediction System 126 (possibly in response to an error found by the user in the designating the one or more ROIs in the WSI or otherwise) to generate a new prediction summary 112 for the adjusted ROI. In another embodiment, a user may also alternatively select specific ROI in the WSI to generate a specific corresponding HER2 score prediction for.

Once the prediction summary 112 is provided to the User Interface and Visualizer 140, the prediction summary 112 may be used by the physician or pathologist users in determining a final HER2 score and status diagnosis for the stained tissue sample captured in the WSI. The prediction summary 112 may also be exported in a portable format or shared with hospital management systems to communicate the prediction summary 112 to a third party for consideration in determining a final HER2 score and status diagnosis for the stained tissue sample captured in the WSI.

While the foregoing is directed to embodiments of the present disclosure, other and further embodiments of the disclosure can be devised without departing from the basic scope thereof, and the scope thereof is determined by the claims that follow.

What is claimed is:

1. A method for generating a predictive HER2 score, comprising:
designating one or more regions of interest in a patient image comprising an IHC stained tissue sample;
identifying a plurality of segments in the patient image using a first machine learning model, wherein each of the plurality of segments comprise nuclei or membrane in the one or more regions of interest;
extracting from the plurality of segments in the patient image a plurality of features based on nuclei or membrane in the plurality of segments;
classifying each of the plurality of features into one of a plurality of feature categories;
generating a predictive HER2 score using a second machine learning model based on the classification of the plurality of features associated with the plurality of segments; and
providing an indication of the predictive HER2 score for the patient image for use in determining a final HER2 score and a HER2 status diagnosis for the IHC stained tissue sample.

2. The method of claim 1, wherein designating one or more regions of interest in the patient image comprises designating one or more regions of interest using a third machine learning model.

3. The method of claim 1, wherein each of the one or more regions of interest comprises a homogenous and contiguous cancer cell population captured in the patient image.

4. The method of claim 1, wherein the first machine learning model comprises a deep-learning neural network machine learning model.

5. The method of claim 1, wherein the second machine learning model comprises one or more of a random forest machine learning model, a support vector machine, a decision tree, a convolutional neural network, or any other machine learning model capable of learning and solving classification problems.

6. The method of claim 1, further comprising partitioning each of the one or more regions of interest of the patient image into a plurality of partitions, wherein each of the plurality of segments identified by the first machine learning model comprises a respective partition of the patient image.

7. The method of claim 1, wherein the plurality of features comprises features corresponding to one or more of the following: an intensity of membrane stain, a completeness of membrane stain, an underlying color of nuclei, an underlying color of membrane, a ratio of membrane and nuclei, a membrane stain deviation, a nuclei stain deviation, an area of completely stained membrane, a stained membrane cell percentage, and a DAB mean color.

8. The method of claim 1, wherein the first machine learning model utilizes shared training weight to identify nuclei and membrane in each of the plurality of segments in the patient image.

9. The method of claim 1, wherein using the second machine learning model to generate the predictive HER2 score for each of the one or more regions of interest further comprises generating the predictive HER2 score based on ASCO/CAP guidelines for HER2 scoring of IHC stained breast cancer tissue cells.

10. The method in claim 7, further comprising generating a heat map identifying the one or more regions of interest and/or nuclei and membrane in each of the one or more regions of interest in the patient image based on the plurality of segments identified by the first machine learning model.

11. A method for training a predictive HER2 tissue scoring model, comprising:
receiving a first training data set including a first plurality of images comprising stained tissue samples;
training a first machine learning model to classify segments of an input image as nuclei or membrane based on the first training data set, wherein the first plurality of images in the first training data set are labeled to identify membrane and nuclei in the plurality of images; and
training a second machine learning model to generate a predictive HER2 score for the input image based on a second training data set, wherein the second training data set comprises a second plurality of images labeled to identify membrane and/or nuclei in the plurality of images, and wherein the second plurality of images are classified in a plurality of categories corresponding to a plurality of features associated with membrane and nuclei in the second plurality of images.

12. The method of claim 11, wherein the first machine learning model comprises a deep-learning neural network machine learning model.

13. The method of claim 11, wherein the plurality of features associated with membrane and nuclei in the second plurality of images comprises features corresponding to one or more of the following: an intensity of membrane stain, a completeness of membrane stain, an underlying color of nuclei, an underlying color of membrane, a ratio of membrane and nuclei, a membrane stain deviation, a nuclei stain deviation, an area of completely stained membrane, and a stained membrane cell percentage.

14. The method of claim 11, wherein the second machine learning model comprises one or more of a random forest machine learning model, a support vector machine, a decision tree, a convolutional neural network, or any other machine learning model capable of learning and solving classification problems.

15. The method of claim 11, wherein training the first machine learning model to classify segments of the input image as nuclei or membrane comprises training the first machine learning model to classify both nuclei and membrane segments utilizing shared training weights.

16. The method of claim 11, wherein training the second machine learning model to generate a predictive HER2 score for the input image based on the second training data set further comprises training the second machine learning model based on the plurality of features relevant to ASCO/CAP guidelines for HER2 scoring of IHC stained breast cancer tissue cells.

17. A non-transitory computer-readable medium storing instruction that, when executed by a processor, cause a computer system to perform the step of:
identifying a plurality of segments in a patient image comprising an IHC stained cancer tissue sample using a first machine learning model, wherein the patient image further comprises one or more regions of interest and each of the plurality of segments comprise nuclei or membrane in the one or more regions of interest;
extracting from the plurality of segments in the patient image a plurality of features based on nuclei or membrane in the plurality of segments;
classifying each of the plurality of features into one of a plurality of feature categories;
generating a predictive HER2 score using a second machine learning model based on the classification of the plurality of features associated with the plurality of segments; and
providing an indication of the predictive HER2 score for the patient image for use in determining a final HER2 score and a HER2 status diagnosis for the stained tissue sample captured in the patient image.

18. The non-transitory computer-readable medium in claim 17, wherein the first machine learning model comprises a deep-learning neural network machine learning model.

19. The non-transitory computer-readable medium in claim 17, wherein the second machine learning model comprises one or more of a random forest machine learning model, a support vector machine, a decision tree, a convolutional neural network, or any other machine learning model capable of learning and solving classification problems.

20. The non-transitory computer-readable medium in claim 17, wherein the plurality of features comprises features corresponding to one or more of the following: an intensity of membrane stain, a completeness of membrane stain, an underlying color of nuclei, an underlying color of membrane, a ratio of membrane and nuclei, a membrane stain deviation, a nuclei stain deviation, an area of completely stained membrane, a stained membrane cell percentage, and a DAB mean color.
